(19) United States
(12) Patent Application Publication
(10) Pub. No.: US 2019/0336433 A1
(43) Pub. Date: Nov. 7, 2019

(54) Hand Sanitizing Composition

(71) Applicant: ESSITY HYGIENE AND HEALTH AKTIEBOLAG, Göteborg (SE)

(72) Inventor: Carolyn Berland, Goteborg (SE)

(21) Appl. No.: 16/310,536

(22) PCT Filed: Jun. 20, 2016
(86) PCT No.: PCT/SE2016/050594

§ 371 Date: Dec. 17, 2018

(51) Int. Cl. A61K 8/92; A61K 8/34; A61K 8/31; A61K 8/60; A61K 8/63; A61Q 19/10

(52) U.S. Cl. CPC A61K 8/922; A61K 8/34; A61K 8/31; A61K 2800/30; A61K 8/60; A61K 8/63; A61Q 19/10; A61K 8/345

(57) ABSTRACT

A hand sanitizing composition including one or more alcohols, a solubilizer, water, and unsaponifiable oil fractions of fatty triglyceride oils. The unsaponifiable oil fractions of fatty triglyceride oils may include a combination of any of the following: squalane, squalene, glycolipids, and phytosterols.

### TECHNICAL BACKGROUND

For hygienic purposes there is a need to clean the hands from bacteria and other harmful microbial entities. Sometimes there is no access to water, where washing with soap and water is not feasible. In other instances, such in hospital care, washing with soap and water is insufficient.

Therefore, the use of hand sanitizers has been developed. The hand sanitizer is usually a mixture of components, where the composition includes an antimicrobial agent. That antimicrobial agent may be benzalkonium chloride or triclosan, but most frequently is some type of alcohol, most often ethanol. Alcohols, being efficient as antimicrobial agents, have some side effects, such as being drying to the skin. Continuous use of such hand sanitizers can leave the user's skin dry, resulting in red, chapped and cracked skin.

To improve the effects the hand sanitizer has on user's skin it has been common to include moisturizers, such as humectants, emollients and the like, into the hand sanitizing composition.

While lending some protection against dry skin, there are several drawbacks with hand sanitizers that include moisturizers. For instance, sanitizers including moisturizers or other hydrophobic skin protectants are unstable and tend to separate over extended periods of time. As a result, the added moisturizers or skin protectants do not remain evenly distributed throughout the sanitizer, rendering the moisturizing ability of the sanitizer ineffective. Additionally, the instability of the sanitizers may cause the formation of large oil layer on the skin and, as a result, the sanitizer may feel greasy and not aesthetically pleasing.

Therefore, the incorporation of moisturizers or skin protectants demands the incorporation of emulsifiers into the sanitizer. However, most emulsifiers are not skin friendly, where the incorporation of the emulsifiers in the sanitizer is counterproductive to adding the moisturizer or skin protectant in the first place.

In view of the problems in known hand sanitizers, we have discovered alcohol-based hand sanitizing compositions that overcome some or all of the problems.

### SUMMARY

It is one object of embodiments of the present invention to provide an alcohol-based hand sanitizing composition. A hand sanitizing composition as described in this invention is a composition which is meant to be applied to the hands or to the skin of the wrists and forearms without the use of additional water. The composition remains on the skin (no-rinse) where it produces a reduction in the number of microorganisms on the hands or skin. The antimicrobial effect can be demonstrated by any of the available standard methods known in the art such as:

Antibacterial effect shown in practical hand washing tests
EN1500
EN12791
ASTM 1174
ASTM 2755

Antibacterial and/or antimicrobial effects demonstrated using suspension tests
EN1275
EN1276
EN13727
EN14476
EN14348
EN1650
ASTM2783

Other tests known in the art can be substituted as desired.

It is a further object of embodiments of the present invention to provide an hand sanitizing composition which does not irritate the skin. For example, the hand sanitizing composition may be substantially free or completely free of emulsifiers.

According to one embodiment of the present invention, it is an object to provide a hand sanitizing composition configured to be dispensed in a sufficiently stable form. Stable form is defined as having the same chemical and haptic properties for the product's defined lifetime.

Further objects and benefits of certain embodiments of the present invention will become apparent from the following description.

Whenever the present description defines a composition as “comprising” certain ingredients, the respective composition may also “consist of” these ingredients in a further embodiment of the present invention. If the terms “optionally” or “may” are used, this indicates that the corresponding ingredient can also be absent from the respective composition. Hereinafter, the unit “wt %” refers to the total weight of the hand sanitizing composition.

An embodiment of the present invention may provide a hand sanitizing composition comprising one or more alcohols, a solubilizer, water, and unsaponifiable oil fractions of fatty triglyceride oils.

According to an embodiment of the present invention the unsaponifiable oil fractions of fatty triglyceride oils may comprise squalane.

According to an embodiment of the present invention the unsaponifiable oil fractions of fatty triglyceride oils may comprise squalene.

According to an embodiment of the present invention the unsaponifiable oil fractions of fatty triglyceride oils may comprise glycolipids.

According to an embodiment of the present invention the unsaponifiable oil fractions of fatty triglyceride oils may comprise phytosterols.

According to an embodiment of the present invention the unsaponifiable oil fractions of fatty triglyceride oils may comprise a combination of any of the following: squalane, squalene, glycolipids, and phytosterols.

According to an embodiment of the present invention the alcohols may comprise ethanol, or isopropanol or a mixture of ethanol and isopropanol.

According to an embodiment of the present invention the solubilizer may be a 3-10 carbon containing diol or triol, more preferably 3-5 carbon containing diol or triol.

According to an embodiment of the present invention the solubilizer may comprise propanediol.

According to an embodiment of the present invention the alcohols may be present in an amount of 65-90 wt %, e.g. 70-80 wt %.

According to an embodiment of the present invention the solubilizer may be present in an amount of 0.5-3 wt %.

According to an embodiment of the present invention the unsaponifiable oil fractions are present in amount of 0.25-2 wt %.

According to an embodiment of the present invention the unsaponifiable oil fractions may comprise 55-70 wt % squalane.

According to an embodiment of the present invention the unsaponifiable oil fractions may comprise 15-25 wt % squalene.

According to an embodiment of the present invention the unsaponifiable oil fractions may comprise 10-15 wt % glycolipids.

According to an embodiment of the present invention the unsaponifiable oil fractions may comprise 1-7 wt % phytosterol.

According to an embodiment of the present invention the unsaponifiable oil fractions may comprise a combination of 55-70 wt % squalane, 15-25 wt % squalene, 10-15 wt % glycolipids and 1-7 wt % phytosterol.

### DETAILED DESCRIPTION

An embodiment of the invention relates to a hand sanitizing composition that is agreeable to the skin. The hand sanitizing composition according to an embodiment of the invention may comprise plant cell oil, for example a plant cell oil obtained from the unsaponifiable fractions of the fatty triglyceride oils. These contain active fractions that play an important role in the integrity and well-being of plants. Present are the fractions which are responsible for cellular adhesion and protection, along with other fractions which play a role in the plant's natural cellular defence system. The chief components are squalene/squalane, which play an integral role in the formation and function of the cellular lipids found in human skin.

The outer layer of the skin consists of a complex mixture of lipophilic and hydrophilic components. These act as a permeability barrier, protecting against invasion of microorganisms as well as preventing moisture loss. Use of soaps and detergents leads to pronounced loss of lipid material from the skin. Moisture is then also lost, which promotes further damage to the permeability barrier, due to its reliance on water for its stability.

The liquid barrier may be replenished from within by release of lipid lamellae from the cellular membrane.

Plant oil is a plant derived solution of phytosterols and glycolipids which will act in a similar way when applied in topical preparations, replenishing those lipids lost from the skin and restoring its suppleness and flexibility.

Squalene is one of the key constituents of the cementing lipids which are responsible for the integrity and healthy appearance of the skin's surface. These lipids interact with the glycolipids present in the skin to help maintain the proper barrier function and skin suppleness and flexibility. They are considered to be natural cell moisturizers found in the cell wall of both plants and animals. The phytosterols may be considered as vegetable lanolin and will impart similar benefits and improvements to the skin.

Plant oil is stable with a very light greaseless feel on the skin. As an emollient, it is a good replacement for animal squalene as it contains additional key moisturizing components of the skin. The stability may be augmented by the addition of tocopherol. The plant oil will synergistically blend with the moisture in the skin, lips or hair.

Since plant oil is a naturally occurring matter, its composition may vary somewhat but it may comprise from 55-70 wt % squalene, 15-25 wt % squalene, 10-15 wt % glycolipids and 1-7 wt % phytosterols.

The solubilizer is preferably a diol. It may be a propanediol, such as commercially available as ZEMEA. It may also be a triol. These solubilizers are advantageous

In that they also provide a moisturizing benefit to the skin.

### BRIEF DESCRIPTION OF THE FIGURES

FIG. 1 shows TEER measurements.

FIG. 2 shows moisturization levels of the skin after application of hand sanitizer.

### EXAMPLES

The following examples illustrate embodiments of the present invention.

### Formulation 1

8 g alcohol (7.2 g ethanol and 0.8 g isopropanol)

50 mg OLEACLEAR OLIVE SQUALANE

2 g water

### Formulation 2

8 g alcohol (7.2 g ethanol and 0.8 g isopropanol)

50 mg OLEACLEAR OLIVE SQUALANE

200 mg ZEMEA

1.75 g water

### Formulation 3

8 g alcohol (7.2 g ethanol and 0.8 g isopropanol)

50 mg PLANELL

200 mg ZEMEA

1.75 g water

### Formulation 4

8 g alcohol (7.2 g ethanol and 0.8 g isopropanol)

50 mg PLANELL

100 mg ZEMEA

1.85 g water

### Formulation 5

8 g alcohol (7.2 g ethanol and 0.8 g isopropanol)

50 mg PLANELL

200 mg Glycerine

1.75 g water

### Formulation 6

8 g alcohol (7.2 g ethanol and 0.8 g isopropanol)

50 mg PLANELL

100 mg Glycerine

1.85 g water

### Formulation 7

8 g alcohol (7.2 g ethanol and 0.8 g isopropanol)

50 mg PLANELL

100 mg isopentyl diol

1.85 g water

Formula 4 is formulated as follows:
1. Add the PLANELL and the ZEMEA to the isopropanol with stirring until dissolved
2. Carefully add the ethanol to the mixture
3. Slowly add the water phase to the ethanol phase with stirring

For all formulas but Formula 4 the blending is done in the following:
1. Pre-mix ethanol and isopropanol
2. Add the other ingredients (except water) to the alcohol with stirring until dissolved
3. Slowly add the water phase to the ethanol phase with stirring

### Example A

Example A discloses the results for testing the antibacterial effect of formulation 4 according to the method EN13727. The results show that the formulation according to the invention has a strong antibacterial effect.

### Test Organism: Pseudomonas aeruginosa NCTC 12924 Lot B2942 Theoretical Concentration: 8.57.10⁷ CFU/Ml

| Real conc. of | | | | Na | | | Contac time |
|---|---|---|---|---|---|---|---|
| the product | Dilution step | V_{c1} | V_{c2} | (=x_{wm} × 10) | Lg Na | Lg R | (min) |
| 97 | 10⁰ | 0 | 0 | <140 | <2.15 | >5.78 | 0.5 |
| | 10⁻¹ | 0 | 0 | <140 | <2.15 | >5.78 | (30 s) |
| | 10⁰ | 0 | 0 | <140 | <2.15 | >5.78 | 1 |
| | 10⁻¹ | 0 | 0 | <140 | <2.15 | >5.78 | |

### Test Organism: Staphylococcus aureus NCTC 10788 Lot B2834 Theoretical Concentration: 9.19.10⁷ CFU/Ml

| Real conc. of | | | | Na | | | Contact time |
|---|---|---|---|---|---|---|---|
| the product | Dilution step | V_{c1} | V_{c2} | (=x_{wm} × 10) | Lg Na | Lg R | (min) |
| 97 | 10⁰ | 0 | 0 | <140 | <2.15 | >5.81 | 0.5 |
| | 10⁻¹ | 0 | 0 | <140 | <2.15 | >5.81 | (30 s) |
| | 10⁰ | 0 | 0 | <140 | <2.15 | >5.81 | 1 |
| | 10⁻¹ | 0 | 0 | <140 | <2.15 | >5.81 | |

### Test Organism: Escherichia coli NCTC 12923 Lot B2710 Theoretical Concentration: 8.87.10⁷ CFU/Ml

| Real conc. of | | | | Na | | | Contact time |
|---|---|---|---|---|---|---|---|
| the product | Dilution step | V_{c1} | V_{c2} | (=x_{wm} × 10) | Lg Na | Lg R | (min) |
| 97 | 10⁰ | 0 | 0 | <140 | <2.15 | >5.8 | 0.5 |
| | 10⁻¹ | 0 | 0 | <140 | <2.15 | >5.8 | (30 s) |
| | 10⁰ | 0 | 0 | <140 | <2.15 | >5.8 | 1 |
| | 10⁻¹ | 0 | 0 | <140 | <2.15 | >5.8 | |

### Example B

Example B shows TEER Measurements (trans-epithelial-electrical-resistance).

TEER measurements shows the movement of ions across the paralcellular pathway regulated by polarized plasma membrane surfaces and by cell-to-cell tight junctions that together prevent movement of solutes and ions across the epithelia. TEER is an indirect assessment of tight junction stability and consequently is a direct measure of the functionality of barrier function in epithelial tissue: it reflects the global resistance of the barrier linked both to the structure and to epithelial thickness. Maintenance of stability and electrical resistance of an epithelium is critical for essential physiological processes, therefore significant changes in TEER may represent an early expression of cell damage.

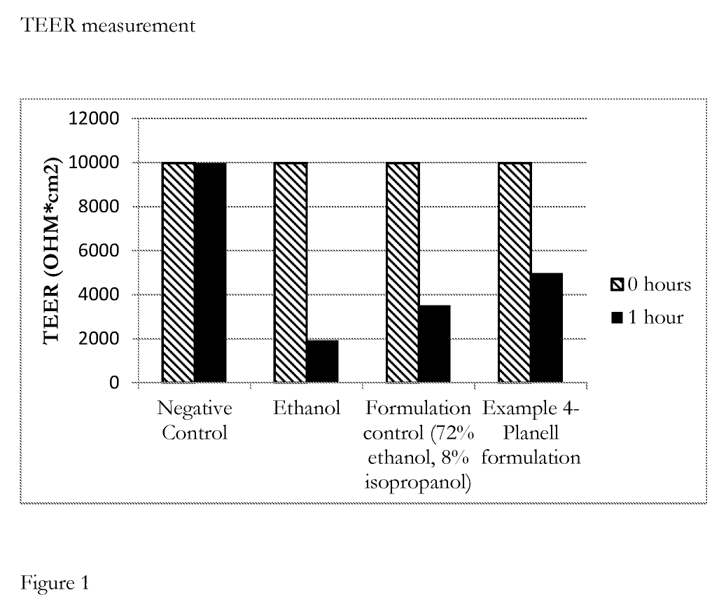

FIG. 1 shows the result of TEER measurements on a negative control, ethanol, a formulation control consisting of 72 wt % ethanol and 8 wt % isopropanol, and on formulation 4. It clearly shows that formulation 4 has less damaging effect on skin than ethanol and the formulation control.

### Example C

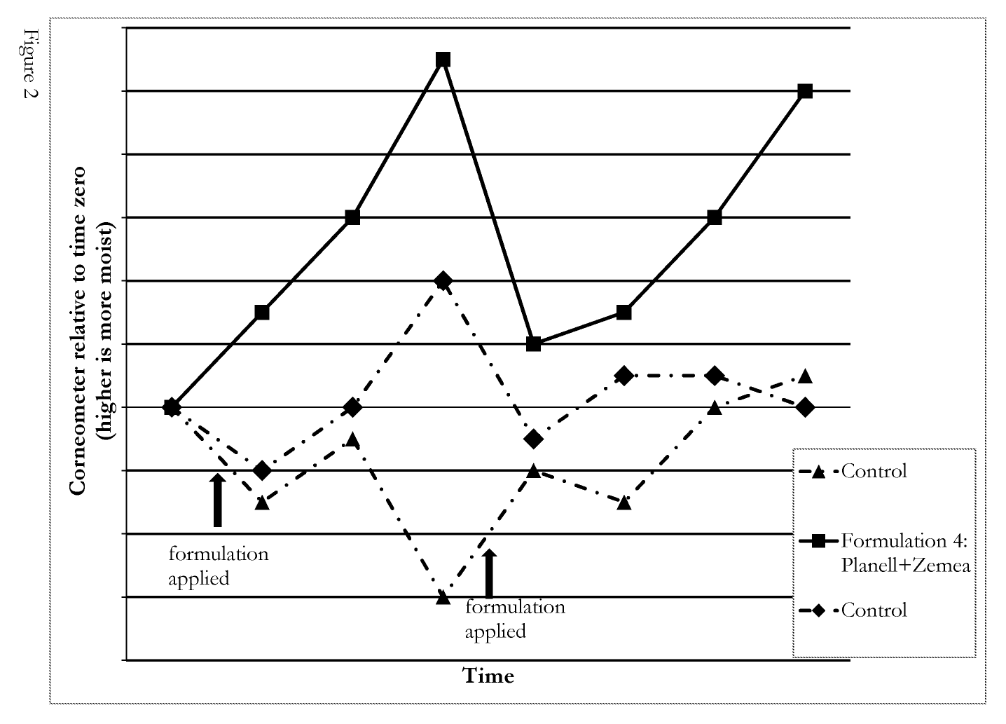

FIG. 2 shows moisturization level of skin of one test person. The test method involved repeated rapid application. The formulation was used to saturate medical gauze pads. The test was carried out by wiping the pad over the test area, allowing alcohol to evaporate and reapplying as soon as the area was dry. The formulation was applied 20 times in succession on day 1 and 15 times in succession on day 2. The condition of the skin was measured: before the application, 1 hour after completed application, at intervals in the following days up to the fourth day. Untreated control sites were used to follow the condition of the skin as this can be expected to vary over the trial. The moisture level on the skin was measured using a corneometer (middle value of three consecutive measurements). Test formulation was applied to upper left forearm (other formulations were tested on lower left and upper and lower right forearm) Control sites were located mid forearm on both right and left arm.

1. A hand sanitizing composition comprising one or more alcohols, a solubilizer, water, and a plant cell oil obtained from the unsaponifiable oil fractions of fatty triglyceride oils;
wherein the hand sanitizing composition is free of emulsifiers, and
wherein the solubilizer is a 3-10 carbon containing diol or triol.

2. A hand sanitizing composition according to claim 1, wherein the unsaponifiable oil fractions of fatty triglyceride oils comprise squalane.

3. A hand sanitizing composition according to claim 1, wherein the unsaponifiable oil fractions of fatty triglyceride oils comprise squalene.

4. A hand sanitizing composition according to claim 1, wherein the unsaponifiable oil fractions of fatty triglyceride oils comprise glycolipids.

5. A hand sanitizing composition according to claim 1, wherein the unsaponifiable oil fractions of fatty triglyceride oils comprise phytosterols.

6. A hand sanitizing composition according to claim 1, wherein the one or more alcohols comprise ethanol, or isopropanol or a mixture of ethanol and isopropanol.

7. A hand sanitizing composition according to claim 1, wherein the solubilizer comprises propandiol.

8. A hand sanitizing composition according to claim 1, wherein the one or more alcohols is present in an amount of 65-90 wt %.

9. A hand sanitizing composition according to claim 1, wherein the solubilizer is present in an amount of 0.5-3 wt %.

10. A hand sanitizing composition according to claim 1, wherein the unsaponifiable oil fractions are present in amount of 0.25-2 wt %.

11. A hand sanitizing composition according to claim 1, wherein the unsaponifiable oil fractions comprise 55-70 wt % squalane.

12. A hand sanitizing composition according to claim 1, wherein the unsaponifiable oil fractions comprise 15-25 wt % squalene.

13. A hand sanitizing composition according to claim 1, wherein the unsaponifiable oil fractions comprise 10-15 wt % glycolipids.

14. A hand sanitizing composition according to claim 1, wherein the unsaponifiable oil fractions comprise 1-7 wt % phytosterol.

15. A hand sanitizing composition according to claim 1, wherein the unsaponifiable oil fractions of fatty triglyceride oils comprise a combination of any of the following: squalane, squalene, glycolipids, and phytosterols.

16. A hand sanitizing composition according to claim 1, wherein the unsaponifiable oil fractions of fatty triglyceride oils comprise a combination of 55-70 wt % squalane, 15-25 wt % squalene, 10-15 wt % glycolipids and 17 wt % phytosterol.
